(19) United States
(12) Patent Application Publication
(10) Pub. No.: US 2021/0278553 A1
(43) Pub. Date: Sep. 9, 2021

(54) THREE-DIMENSIONAL SOLID STATE IMAGING PHOTODETECTOR

(71) Applicant: KONINKLIJKE PHILIPS N.V., EINDHOVEN (NL)

(72) Inventors: Marc Anthony CHAPPO, ELYRIA, OH (US); Douglas B. MCKNIGHT, CHARDON, OH (US)

(21) Appl. No.: 16/317,841

(22) PCT Filed: Jul. 31, 2017
(86) PCT No.: PCT/EP2017/069335

§ 371 Date: Jan. 15, 2019

(51) Int. Cl. G01T 1/20; A61B 6/03; G01N 23/046; A61B 6/00; G01N 23/083

(52) U.S. Cl. CPC G01T 1/2018; A61B 6/032; G01N 23/046; G01N 2223/505; G01N 23/083; G01N 2223/04; G01N 2223/419; A61B 6/4208

(57) ABSTRACT

A detector array (112) includes a detector pixel (206). The detector pixel includes a three dimensional cavity (304 and 306; 432 and 404) having walls (308/602 and 316; 434 and 406/502) that include active regions, which detect light photons traversing within the three dimensional cavity and produce respective electrical signals indicative thereof. The detector pixel further includes a first scintillator (320; 410) disposed in the three dimensional cavity adjacent to a bottom (320; 416) of the at least one detector pixel. The detector pixel further includes a second scintillator (326; 444) disposed in the three dimensional cavity on top of the first scintillator, wherein the first and second scintillators emits the light photons in response to absorbing x-ray photons. At least one of the walls is vertically oriented with respect to detector pixel, maximizing contact area between a corresponding active region and one of the first or second scintillators.

### FIELD OF THE INVENTION

The following generally relates to an imaging detector and more particularly to a three dimensional (3-D) solid state imaging photodetector and is described in connection with computed tomography (CT), including medical and/or baggage CT scanners. However, the following is also amenable to other imaging modalities and/or imaging applications.

### BACKGROUND OF THE INVENTION

A computed tomography (CT) scanner generally includes an X-ray tube mounted on a rotatable gantry that rotates around an examination region about a z-axis. The X-ray tube emits radiation that traverses the examination region and a subject or object positioned therein. An X-ray sensitive radiation detector array subtends an angular arc opposite the examination region from the X-ray tube, detects radiation that traverses the examination region, and generates a signal indicative thereof. A reconstructor processes the signal and reconstructs volumetric image data indicative thereof the examination region and the portion of the subject or object therein during scanning.

Such a detector array has included crystal or garnet scintillators directly mounted to flat solid-state photodetectors such as photodiodes. The scintillator material produces light photons in response to bombardment with X-Ray photons, which are then converted to electrical currents or pulses in the photodetector. However, response time and efficiency of collection of charge carriers in photodetectors are related to the geometry of today's flat X-ray sensitive radiation detector arrays, as well as the interaction between the scintillators silicon detectors that generate charge carriers in response to photons.

US 2015/0276939 A1 to Chappo et al., which is incorporated by reference in its entirety herein, describes an X-ray sensitive radiation detector array with a third dimension of depth. The geometry of this 3-D detector array improves charge collection efficiency relative to a two-dimensional (2-D) flat photodetector. Unfortunately, charge collection inefficiencies result in a patient being irradiated with ionizing radiation that does not contribute to an image, and ionizing radiation can cause damage to tissue which can result in numerous health issues. As such, there is an unresolved need further improvement in charge collection efficiency.

### SUMMARY OF THE INVENTION

Aspects described herein address the above-referenced problems and/or others.

In one aspect, a detector array includes a detector pixel. The detector pixel includes a three dimensional cavity having walls that include active regions, which detect light photons traversing within the three dimensional cavity and produce respective electrical signals indicative thereof. The detector pixel further includes a first scintillator disposed in the three dimensional cavity adjacent a bottom of the at least one detector pixel. The detector pixel further includes a second scintillator disposed in the three dimensional cavity on top of the first scintillator, wherein the first and second scintillators emits the light photons in response to absorbing x-ray photons. At least one of the walls is vertically oriented with respect to detector pixel, maximizing contact area between a corresponding active region and one of the first or second scintillators.

In another aspect, a method includes receiving X-ray photons with scintillators of a three-dimensional solid state imaging photodetector, absorbing, with the scintillators, the X-ray photons, and producing, with the scintillators and in response to absorbing the X-ray photons, light photons indicative of an energy of the X-ray photons. The method further includes sensing the light photons with active regions of the three-dimensional solid state imaging photodetector, and producing, with active regions and in response to detecting the light photons, an electrical signal indicative of the energy of the X-ray photons. A contact area between the scintillator and the active areas is maximized.

In another aspect, an imaging system includes an X-ray source configured to emit X-rays, a three-dimensional solid state imaging photodetector configured to detect X-rays and generate a signal indicative thereof, and a reconstructor configured to reconstruct the signals from the detector. The three-dimensional solid state imaging photodetector includes first and second scintillators disposed in one or more recesses of active areas such that a contact area between one of the first and second scintillators and a wall of an active area is maximized.

The invention may take form in various components and arrangements of components, and in various steps and arrangements of steps. The drawings are only for purposes of illustrating the preferred embodiments and are not to be construed as limiting the invention.

### BRIEF DESCRIPTION OF THE DRAWINGS

FIG. 1 diagrammatically illustrates an example imaging system with 3-D solid state imaging photodetector.

FIG. 2 diagrammatically illustrates an example detector tile of the 3-D solid state imaging photodetector.

FIG. 3 diagrammatically illustrates an example pixel of the detector tile.

FIG. 4 diagrammatically illustrates another example pixel of the detector tile.

FIG. 5 diagrammatically illustrates yet another example pixel of the detector tile.

FIG. 6 diagrammatically illustrates still another example pixel of the detector tile.

FIG. 7 illustrates an example method in accordance with an embodiment herein.

### DETAILED DESCRIPTION OF EMBODIMENTS

FIG. 1 diagrammatically illustrates an imaging system 100 such as a computed tomography (CT) scanner.

The imaging system 100 includes a generally stationary gantry 102 and a rotating gantry 104. The rotating gantry 104 is rotatably supported by the stationary gantry 102 by a bearing (not visible) or the like and rotates around an examination region 106 about a longitudinal or z-axis. A radiation source 108, such as an X-ray tube, is supported by and rotates with the rotating gantry 104, and emits X-ray radiation. A collimator 109 collimates the radiation, producing a generally cone, fan, wedge, or otherwise shaped radiation beam that traverses the examination region 106.

A radiation sensitive detector array 112 subtends an angular arc opposite the radiation sources 108 across the examination region 106 and detects radiation traversing the examination region 106 and generates and outputs an electrical signal or pulse indicative thereof. The radiation sensitive detector array 112 includes one or more rows of detector tiles 114 arranged along a z-direction. U.S. Pat. No. 6,510,195 to Chappo et al., which is incorporated by reference in its entirety herein, describes an example of a suitable detector tile. Optionally, a focused or non-focused anti-scatter grid (ASG) can be employed with the radiation sensitive detector array 112.

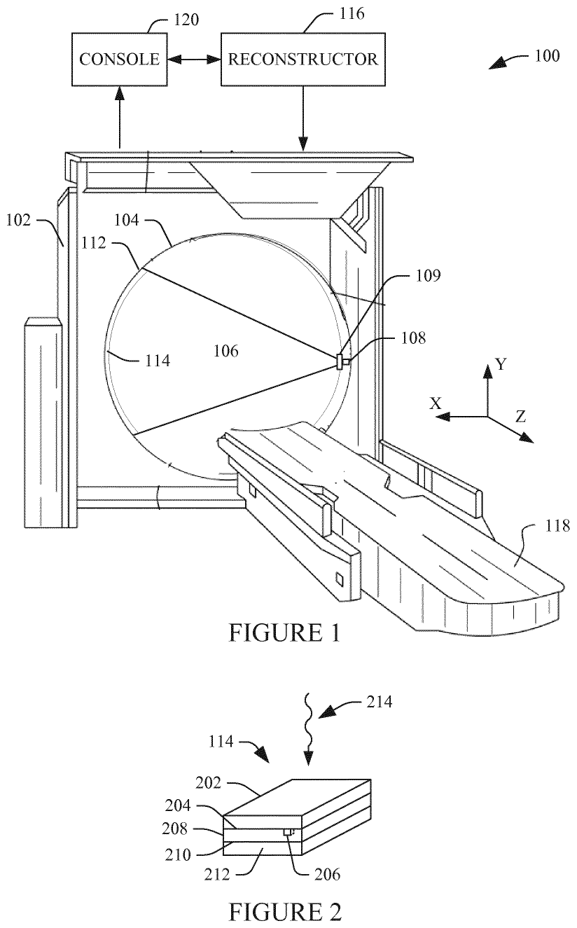

Briefly turning to FIG. 2, a non-limiting example of the detector tile 114 is schematically illustrated. The relative geometry (i.e., shape, size, etc.) of the detector tile 114 is not limiting. The detector tile 114 includes a scintillator layer 202 (e.g., including one or multiple scintillators, at least two having a same or a different X-ray absorption characteristic) that is optically coupled to a photosensitive side 204 of a photosensitive layer 208. The photosensitive layer 208 has a plurality of active regions or photosensitive pixels 206 (only one shown for sake of clarity). A non-photosensitive side 210 of the photosensitive layer 208 is electrically coupled to a substrate 212, which includes readout electronics such as an ASIC, and/or other circuitry.

In one non-limiting instance, the photosensitive layer 208 and the photosensitive pixels 206 contain or are composed of silicon (Si). Non-active regions of the photosensitive layer 208 include electrodes that inter-connect each detector pixel to an electrical contact. The substrate 212 includes a silicon or other ASIC bonded to the non-photosensitive area of the silicon photosensitive layer 208 and in electrical communication with the electrical contacts. A non-limiting example of such a silicon detector is described in U.S. patent application publication 2009/0121146 to Luhta et al., which is incorporated herein by reference in its entirety.

The photosensitive pixel 206 includes a cavity that defines a 3-D volume and that includes a 3-D surface with multiple active regions, wherein at least a sub-portion of the scintillator layer 208 is disposed in the cavity and light photons emitted therein are detected in three dimensions by the multiple active regions. As described in greater detail below, in one instance, a shape of the cavity maximizes a contact area between the scintillator layer 208 and the active regions and reduces a distance between the active regions and readout electronics.

Returning to FIG. 1, a reconstructor 116 reconstructs the output signal output and generates volumetric three-dimensional image data. Where the detector tile 114 is configured as a multi-energy detector (e.g., including multiple scintillators, each having a different X-ray absorption characteristic), this includes generating spectral images and/or conventional non-spectral images. A subject support 118, such as a couch, supports a subject or object in the examination region 106. A general purpose computing system serves as an operator console 120, which includes human readable output devices such as a display and/or printer and input devices such as a keyboard and/or mouse. Software resident on the console 120 allows the operator to control an operation of the imaging system 100.

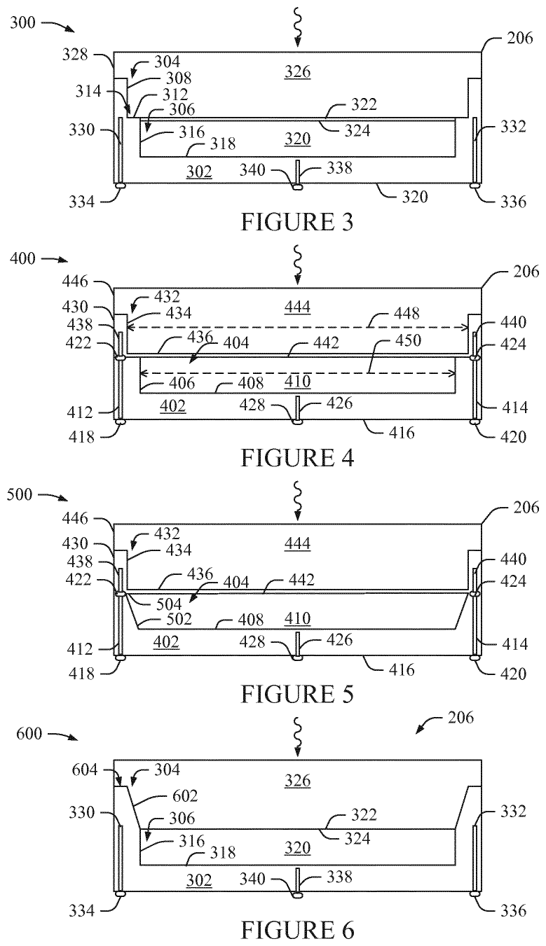

FIG. 3 diagrammatically illustrates a cross-sectional view of an embodiment 300 of an example of the photosensitive pixel 206. The photosensitive pixel 206 includes a single block 302 of silicon with a first recess 304 and a second recess 306 within the first recess 304. The first recess 304 includes generally planar walls 308, which are generally vertical with respect to the photosensitive pixel 206. The first recess 304 further includes a generally planar floor 312, which is horizontal with respect to the photosensitive pixel 206 and which is generally perpendicular to the planar walls 308 from which the planar walls 308 extend.

The second recess 306 is in a sub-portion of the floor 312 of the first recess 304. That is, as shown, the floor 312 extends from the planar walls 308 a non-zero, finite distance towards a center region of the pixel 206 to the second recess 306, forming a “ledge” region 314. In a variation, the distance is about zero and there is no ledge region 314. The second recess 306 includes generally planar walls 316, which are generally vertical with respect to the photosensitive pixel 206. The second recess 306 further includes generally planar floor 318, which is horizontal with respect to the photosensitive pixel 206 and which is generally perpendicular to the planar walls 316 from which the planar walls 316 extend. The first and second recesses 304 and 306 define a 3-D cavity.

A first scintillator 320 is disposed in the second recess 306. An optical coating 322 is disposed over a first surface 324 of the first scintillator 320, which is opposite the floor 318. The optical coating 322 reflects light photons, which may improve light collection efficiency, and passes X-ray photons. In a variation, the optical coating 322 is omitted, more than one optical coating is utilized, etc. A second scintillator 326 is disposed in the first recess 304 over the optical coating 322 and the ledge region 314. The second scintillator 326 includes sub-portions 328 that extend over the planar walls 308.

First and second electrically conductive paths 330 and 332 extend from an active area associated with the side surface of 326 of the pixel 206 and along the planar walls 316 to the planar walls 308 at opposing ends of the pixel 206. First and second electrodes 334 and 336 are located near the bottom surface 320 respectively in electrical contact with the first and the second electrode 336. A third electrically conductive path 338 extends from the active area at the bottom surface 320 into the block 302 under the first scintillator 320 and is in electrical contact with a third electrode 340. The conductive paths 330, 332 and 338 are disposed, e.g., in Through Si Vias (TSVs) and/or other technology. More conductive paths than the minimum amount shown may be included.

With this configuration, the active area of the silicon block 302 and the scintillators 320 and 326 are configured and oriented with respect to each other to maximize contact surface between them, improving charge collection efficiency relative to a configuration without these feature. Furthermore, the conductive paths 330 and 332 in close proximity the walls 308 and surface 314, and the conductive path 338 is in close proximity the surface 318, reducing carrier transport and collection time, relative to a configuration without these features.

In one instance, the first scintillator 320 has a first x-ray absorption characteristic, and the second scintillator 326 has a second X-ray absorption characteristic, where the first and second x-ray absorption characteristics are different. For example, in this instance the first scintillator 320 absorbs X-rays having energy in a first range, and the second scintillator 326 absorbs X-rays having energy in a second range, wherein the first and second ranges are different. Such a configuration is well-suited for multi-energy imaging. In another instance, the first and second x-ray absorption characteristics are the same.

FIG. 4 diagrammatically illustrates a cross-sectional view of an embodiment 400 of an example of the photosensitive pixel 206. The photosensitive pixel 206 includes a first block 402 of silicon with a first recess 404. The first recess 404 includes generally planar walls 406, with respect to the photosensitive pixel 206. The second recess 404 further includes generally planar floor 408, which is horizontal with respect to the photosensitive pixel 206 and which is generally perpendicular to the planar walls 406 from which the planar walls 406 extend.

A first scintillator 410 is disposed in the first recess 404. First and second electrically conductive paths 412 and 414 extend from a bottom surface 416 of the pixel 206 and along the planar walls 406 at opposing ends of the pixel 206. First and second electrodes 418 and 420 are located at the bottom surface 416 respectively in electrical contact with the first and the electrically conductive paths 412 and 414. Third and fourth electrodes 422 and 424 are located opposing ends of the electrically conductive paths 412 and 414. A fifth electrically conductive path 426 extends from the bottom surface 416 into the block 402 under the first scintillator 410 and is in electrical contact with a fifth electrode 428.

The photosensitive pixel 206 includes a second block 430 of silicon with a second recess 432, which includes generally planar walls 434, which are generally vertical with respect to the photosensitive pixel 206. The second recess 434 further includes a generally planar floor 436, which is horizontal with respect to the photosensitive pixel 206 and which is generally perpendicular to the planar walls 434. Third and fourth electrically conductive paths 438 and 440 are in the second block 430 along the planar walls 434 at opposing ends of the pixel 206. A second scintillator 444 is disposed in the second recess 432 and includes portions 446 that extend over the planar walls 434.

In this embodiment, the second block 430 of silicon is stacked on top of the first block 402 of silicon over the first recess 404 with the third and fourth electrically conductive paths 438 and 440 in electrical contact with the third and fourth electrodes 422 and 424. The second block 430 is mounted or otherwise permanently or removably affixed to the first block 402. In this example, a first width 448 of the first recess 404 is larger than a second width 450 of the second recess 432. In a variation, the first and second widths are the same. It is to be appreciated that the bottom portion 442 of the top Si portion 446 is an active collection area.

Similar to the example in FIG. 3, with this configuration, the first and second blocks 402 and 430 are configured and oriented with respect to each other to maximize contact surface between scintillators and active surfaces of the silicon, improving charge transport time and collection efficiency relative to a configuration without these feature. Furthermore, the conductive paths 438 and 440 in close proximity the walls 434, and the conductive path 426 is in close proximity the surface 408, reducing carrier collection time, relative to a configuration without these features. Likewise, in this example the pixel 206 can be a single or multi-energy pixel.

FIG. 5 diagrammatically illustrates a cross-sectional view of an embodiment 500 of a photosensitive pixel 206. This embodiment is substantially similar to the embodiment 400 described in FIG. 4, except that the first recess 404 in the first block 402 of silicon includes generally planar walls 502, which are generally transverse with respect to the photosensitive pixel 206.

For example, in the illustrated embodiment the planar walls 502 extend from a top 504 of the first block 402 to the surface 408 of the first recess 410 at an angle in a range of forty-five degrees (45°) to sixty degrees (60°) such as fifty-six degrees (56°). The vertical walls 434 can be formed via ion etch and/or other technology, and the transverse walls 502 can be formed via chemical etching and/or other technology.

This configuration reduces carrier collection time and improves photon and charge collection efficiency, relative to a configuration with a 2-D flat detector. Likewise, in one instance the X-ray absorption characteristics of the first and second scintillator are different, and in another instance the X-ray absorption characteristics of the first and second scintillator are the same.

FIG. 6 diagrammatically illustrates a cross-sectional view through of an embodiment 600 of a photosensitive pixel 206. This embodiment is substantially similar to the embodiment 300 described in FIG. 3, except that the first recess 304 in the block 302 includes generally planar walls 602, with respect to the photosensitive pixel 206.

For example, in the illustrated embodiment the planar walls 602 extend from a top 604 of the block 302 to the second recess 306 at an angle in a range of forty-five degrees (45°) to sixty degrees (60°) such as fifty-six degrees (56°). The vertical walls 316 can be formed via ion etch and/or other technology, and the transverse walls 602 can be formed via chemical etching and/or other technology.

This configuration reduces carrier collection time and improves photon and charge collection efficiency, relative to a configuration with a 2-D flat detector. Likewise, in one instance the X-ray absorption characteristics of the first and second scintillator are different, and in another instance the X-ray absorption characteristics of the first and second scintillator are the same.

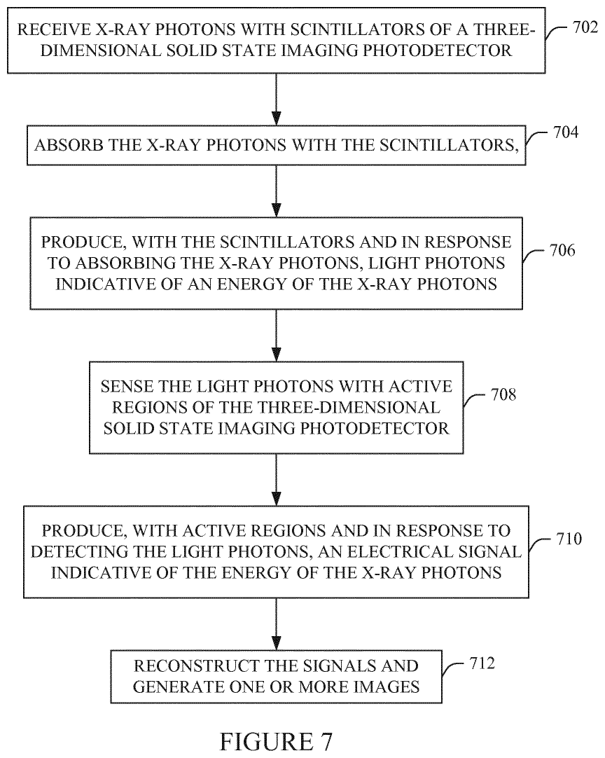

FIG. 7 illustrates an example method in accordance with an embodiment herein.

At 702, X-ray photons are received by the scintillator layer 202 (FIG. 2) of the three-dimensional solid state imaging photodetector 114 (FIGS. 1 and 2).

At 704, the one or more of the scintillators 320, 326, 410 and/or 444 (FIGS. 3-6) absorb the X-ray photons.

At 706, the one or more of the scintillators 320, 326, 410 and/or 444, in response to absorbing the X-ray photons, emit light photons indicative of an energy of the X-ray photons.

At 708, one or more of the active regions 308, 316, 318 434, 406, 408 (FIGS. 3-6) of the three-dimensional solid state imaging photodetector 114 sense the light photons.

As described herein, a contact area between the one or more of the scintillators 320, 326, 410 and/or 444 and the one or more of the active regions 308, 316, 318 434, 406, 408 is maximized, or at least increased over 2-D flat detectors and/or other 3-D detectors, and/or distances between the contacts of one or more of the active regions 308, 316, 318 434, 406, 408 and readout conductive paths are shortened relative to other 3-D detectors, thus improving charge transport and collection times.

At 710, the one or more of the active regions 308, 316, 318 434, 406, 408 produce, in response to detecting the light photons, an electrical signal indicative of the energy of the X-ray photons.

At 712, the reconstructor 116 (FIG. 1) reconstructs the signals and generates one or more images.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A detector array, comprising:
a detector pixel, including:
a three dimensional cavity having walls that include active regions, which detect light photons traversing within the three dimensional cavity and produce respective electrical signals indicative thereof;
a first scintillator disposed in the three dimensional cavity adjacent to a bottom of the detector pixel; and
a second scintillator disposed in the three dimensional cavity on top of the first scintillator, wherein the first and second scintillators emits the light photons in response to absorbing x-ray photons,
wherein at least one of the walls is vertically oriented with respect to detector pixel, maximizing contact area between a corresponding active region and one of the first or second scintillators.

2. The detector array of claim 1, wherein the three dimensional cavity includes a first recess and a second recess within the first recess, the first scintillator is disposed in the second recess, and the second scintillator is disposed in the first recess, and each of the first and second recesses includes only vertically oriented walls.

3. The detector array of claim 2, wherein the at least one detector pixel further includes an optical layer disposed between the first and second scintillators.

4. The detector array of claim 1, wherein the three dimensional cavity includes a first recess and a second recess within the first recess, the first scintillator is disposed in the second recess, the second scintillator is disposed in the first recess, the first recess includes a transverse wall, and the second recess includes only vertically oriented walls.

5. The detector array of claim 2, further comprising:
electrodes disposed at a side of the detector pixel;
vias extending from the active areas to the electrodes; and
electrically conductive paths disposed in the vias from the active areas to the electrodes.

6. The detector array of claim 5, wherein the detector pixel comprises of a single block of silicon.

7. The detector array of claim 1, wherein the at least one detector pixel further includes at least two blocks, including a first block with a first recess in which the first scintillator is disposed, and a second block with a second recess in which the second scintillator is disposed.

8. The detector array of claim 7, wherein the first and second blocks are coupled together.

9. The detector array of claim 7 wherein the first block includes first and second electrically conductive paths, and the second block includes third and fourth electrically conductive paths, and the first and second electrically conductive paths are in electrical contact with the includes third and fourth electrically conductive paths.

10. The detector array of claim 7, wherein the first recess includes a transverse wall, and the second recess includes only vertically oriented walls.

11. The detector array of claim 1, wherein the three dimensional cavity includes a first recess having a floor extending towards a center region of the pixel to a second recess in the first recess, the floor providing a ledge region between the walls of the first and second recesses, the first scintillator is disposed in the second recess, and the second scintillator is disposed in the first recess.

12. The detector array of claim 1, wherein the first scintillator has a first X-ray absorption characteristic and the second scintillator has a second X-ray absorption characteristic, and the first and second X-ray absorption characteristics are different.

13. The detector array of claim 1, wherein the first scintillator has a first X-ray absorption and the second scintillator has a second X-ray absorption characteristic, and the first and second X-ray absorption characteristics are the same.

14. A method, comprising:
receiving X-ray photons with scintillators of a detector pixel;
absorbing, with the scintillators, the X-ray photons;
producing, with the scintillators and in response to absorbing the X-ray photons, light photons indicative of an energy of the X-ray photons;
sensing the light photons with active regions of the detector pixel,
wherein a contact area between the scintillators and the active areas is maximized; and
producing, with active regions and in response to detecting the light photons, an electrical signal indicative of the energy of the X-ray photons.

15. The method of claim 14, further comprising:
detecting photons having first energy with a first of the scintillators;
detecting photons having second different energy with a second different one of the scintillators; and
reconstructing the electrical signal to generate a spectral image.

16. An imaging system, comprising:
an X-ray source configured to emit X-rays;
a detector pixel configured to detect X-rays and generate a signal indicative thereof, wherein the detector pixel includes first and second scintillators disposed in one or more recesses of active areas such that a contact area between one of the first and second scintillators and a wall of an active area is maximized; and
a reconstructor configured to reconstruct the signals from the detector.

17. The imaging system of claim 16, wherein detector pixel includes a single block of silicon and all of walls of the actives areas are vertical.

18. The imaging system of claim 16, wherein the detector pixel includes at least two blocks of silicon, one supporting the first scintillator, and another supporting the second scintillator, and all of walls of the actives areas are vertical.

19. The imaging system of claim 16, wherein the detector pixel includes a single block of silicon, one of the walls of the actives areas is vertical, and another of the walls of the actives areas is transverse.

20. The imaging system of claim 16, wherein the detector pixel includes at least two blocks of silicon, one supporting the first scintillator, and another supporting the second scintillator, one of the walls of the actives areas is vertical, and another of the walls of the actives areas is transverse.
